(19) United States
(12) Patent Application Publication
(10) Pub. No.: US 2012/0148638 A1
(43) Pub. Date: Jun. 14, 2012

(54) Steroid Solavates

(75) Inventors: Ian Cameron Gardner McAffer, Kent (GB); Peter Ernest Tasko, Stevenage (GB); Susheela Ginafrancesco, Stevenage (GB); Graham John Swift, Stevenage (GB)

(73) Assignee: BREATH LIMITED, Stevenage (GB)

(21) Appl. No.: 13/390,226

(22) PCT Filed: Aug. 16, 2010
(86) PCT No.: PCT/EP2010/061916

§ 371 Date: Feb. 13, 2012

(30) Foreign Application Priority Data
Aug. 14, 2009 (GB) 0914240.7

(51) Int. Cl. A61K 9/14; A61K 31/573; A61P 11/00; C07J 5/00

(52) U.S. Cl. 424/400; 552/574; 514/171; 514/180; 428/402

(57) ABSTRACT

A dpi formulation comprises a solvate of beclomethasone with a non-cyclic, straight or branched C₅-C₇ hydrocarbon. The solvate particles are of size 0.5 to 10 microns and are obtained by crystallization of the steroid in the presence of ultrasound.

### FIELD

The present invention relates to formulations of steroid solvates, in particular solvates of beclomethasone. Preferred formulations of the invention are for use in dry powder inhalers.

### BACKGROUND

Delivery of steroids to the lungs via aerosol is widely known and used for the treatment of a number of diseases, including asthma, airways disease and chronic obstructive pulmonary disease (COPD). Formulations are generally administered via dry powder inhaler (dpi), metered dose inhaler (mdi) and, to a lesser extent, nebuliser.

### PROBLEMS

A nebuliser formulation of beclomethasone is known, containing a suspension of beclomethasone diproprionate particles falling in the size range 2-5 microns. This formulation is successfully used for administration of beclomethasone, with apparently suitable particle size and particle size distribution. However, the inventors hereof have identified a problem with the formulation, namely that it does not store well, any period of storage tending to result in product settling in the container, e.g. in the bottom of the ampoule or if inverted in the head, and being difficult to resuspend even after significant agitation; this resuspension is, to the inventors, notably more difficult than with other steroids used in nebuliser formulations.

In addition, a general problem associated with dpi formulations is that the effective dose of beclomethasone at point of delivery to the patient is rather lower than that contained in the formulation, it being acknowledged that a certain loss of product occurs during delivery, though again, to the inventors, the amount lost is higher than for other steroids. This loss is compensated for in the amount of active included in the formulation, a solution regarded as acceptable. Nevertheless, it would be desirable to reduce this loss: any loss is to some degree uncontrollable and hence affects the reliability of dosing.

A method of preparing small crystals is described in WO 02/089942, in which crystallization occurs in the presence of ultrasound. WO 2004/073827 describes preparation of aerosol formulation for mdi and dpi uses, again using ultrasound during crystallization of the active component. WO 2010/007447 describes a process for increasing the crystallinity of a solid material and describes the use of the process for preparing particles for dpi formulations.

An aim of the present invention is to provide alternative, preferably improved steroid formulations and methods of making the same, in particular, steroid formulations that exhibit less loss of product in use.

### INVENTION

Accordingly, the invention provides a solvate of (i) beclomethasone and (ii) a C₅-C₇ hydrocarbon. The C₅-C₇ hydrocarbon is typically non-cyclic, straight or branched and in a preferred embodiment of the invention the C₅-C₇ hydrocarbon is heptane. In a specific embodiment the heptane is n-heptane.

The solvates of the invention can advantageously be used in preparation of pharmaceutical formulations, with reduced loss of active during delivery and being stable in storage.

A suitable steroid for formation of the solvate of the invention is beclomethasone. In a preferred embodiment of the invention the solvate is beclomethasone-heptane.

Typically the solvate of the invention is in crystalline form and is obtained by crystallisation in the presence of ultrasound.

In use of the invention, solvate particles are obtained which are approximately rounder, spherical particles. Solvate particles of the invention are characterized by a regular shape and smooth surface morphology.

Preferred solvates of the invention comprise solvate particles of size 0.5-10 microns, more preferably 0.5-5 microns. Further it is preferred that a substantial proportion of the product be within these stated size ranges so that a substantial proportion will reach the patient's lungs, and preferably at least 75% (by number), more preferably at least 90% (by number) of the solvate particles are within the stated size range. It is further preferred that the solvate particle size distribution is within the range X₁₀=>1.0 microns, X₅₀=<5 microns and X₉₀=<10 microns, more preferably within the range X₁₀=>0.5 microns, X₅₀=<3 microns and X₉₀=<7 microns and in a particular embodiment of the invention the particle size distribution is within the range X₁₀=>0.5 microns, X₅₀=<3 microns and X₉₀=<5 microns.

Particle size or particle diameter as used herein can be suitably determined by laser diffraction based methods, for example as described in ISO Standard 13320-1. Laser diffraction particle sizing apparatus such as the Malvern Mastersizer 2000™ can be used.

The present invention also provides a dpi formulation, comprising a solvate of (i) beclomethasone and (ii) a C₅-C₇ hydrocarbon. The C₅-C₇ hydrocarbon is typically non-cyclic, straight or branched and in a preferred embodiment of the invention the C₅-C₇ hydrocarbon is heptane. In a specific embodiment the heptane is n-heptane.

In an alternative embodiment of the invention the dpi formulation comprises a solvate of (i) beclomethasone and (ii) water. In a preferred embodiment of the invention the solvate is beclomethasone monohydrate.

A suitable steroid for formation of the solvate for use in the formulation is beclomethasone. In a preferred embodiment of the formulation the solvate is beclomethasone-heptane. In an alternative embodiment of the invention the solvate is beclomethasone monohydrate.

Formulations of the invention can comprise 0.1% to 10% (by weight) of beclomethasone solvate, preferably 0.2% to 8% (by weight); more preferably formulations of the invention comprise 0.3% to 6% (by weight) of beclomethasone solvate.

Suitable dpi formulations of the invention can comprise individual doses of about 25 mcg of beclomethasone and about 1.25 mg of carrier, in another embodiment of the invention the formulations comprise individual doses of about 50 mcg of beclomethasone and about 2.5 mg of carrier and in a yet further embodiment of the invention the formulations comprise individual doses of about 125 mcg of beclomethasone and about 6.35 mg of carrier. Alternatively, dpi formulations of the invention can comprise individual doses of about 37.5 mcg of beclomethasone and about 1.25 mg of carrier, in another embodiment of the invention the formulations comprise individual doses of about 75 mcg of beclomethasone and about 2.5 mg of carrier and in a yet further embodiment of the invention the formulations comprise individual doses of about 187.5 mcg of beclomethasone and about 6.35 mg of carrier.

Typically the solvate for use in the formulation is in crystalline form and is obtained by crystallisation in the presence of ultrasound.

Preferred solvates for use in the formulation of the invention comprise solvate particles of size 0.5-10 microns, more preferably 0.5-5 microns. Further it is preferred that a substantial proportion of the product be within these stated size ranges so that a substantial proportion will reach the patient's lungs, and preferably at least 75% (by number), more preferably at least 90% (by number) of the solvate particles are within the stated size range. It is further preferred that the solvate particle size distribution is within the range X₁₀=>0.1 microns, X₅₀=<5 microns and X₉₀=<10 microns, more preferably within the range X₁₀=>0.5 microns, X₅₀=<3 microns and X₉₀=<7 microns and in a particular embodiment of the invention the particle size distribution is within the range X₁₀=>0.5 microns, X₅₀=<3 microns and X₉₀=<5 microns.

In use of formulations of the invention typically at least 80% (by weight) of the dose of active is delivered to the patient, preferably at least 85% (by weight) and more preferably at least 90% (by weight) of the dose of active is delivered to the patient. In a particular embodiment of the invention at least 95% (by weight) of the dose of active is delivered to the patient and in a further embodiment of the invention at least 97% (by weight) can be delivered to the patient. In a yet further embodiment of the invention up to 98% (by weight) of the dose of active is delivered to the patient. In alternative embodiments of the invention up to 99% (by weight) or up to 100% (by weight) of the dose of active can be delivered to the patient.

The dpi formulation of the invention can further comprise a beta-agonist. Suitable beta-agonists for use in the formulation include salbutamol, levalbuterol and formoterol.

In a specific embodiment of the invention the formulation further comprises a carrier. Preferred carriers for use in the formulation of the invention include lactose, mannitol, glucose and polyethylene glycol. Formulations of the invention can comprise 80% to 99.9% (by weight) of carrier, preferably 90% to 99.9% (by weight); more preferably formulations of the invention comprise 94% to 99.7% (by weight) of carrier.

The present invention also provides a method of preparing a solvate of beclomethasone, comprising forming a suspension of (i) droplets containing beclomethasone dissolved in a solvent, in (ii) a C₅-C₇ hydrocarbon non-solvent of beclomethasone, and applying ultrasound to the droplets to form the solvate. Typically the solvate of the invention is in crystalline form.

In an alternative embodiment of the invention, the method comprises drying a solution of beclomethasone in a solvent to obtain solid, preferably substantially amorphous particles, which are then contacted with the C₅-C₇ hydrocarbon non-solvent of beclomethasone and subjected to ultrasound to form crystallized beclomethasone particles. The solution may be in the form of droplets. The drying may be carried out by rapid precipitation, freeze drying, lyophilisation, rapid expansion of supercritical solution, spray drying or mixtures thereof.

Beclomethasone is suitably crystallized by forming a solution of steroid in a solvent, forming a suspension of droplets of the solution in a non-solvent of the steroid, and applying ultrasound to the droplets. The steroid in the suspended droplets, which may be mainly or entirely beclomethasone, crystallizes to form particles of a generally spherical type. More specifically, it is crystallized by dissolving it in a solvent, forming droplets of the solution, for example by generating an aerosol from this solution, forming a dispersion of the droplets in a non-solvent of the steroid and subjecting the droplets to ultrasound to initiate or effect crystallization of the steroid.

Droplets can be prepared by electrohydrodynamic spraying, atomizing using high pressure, spray nozzles, nebulisers, transducers such as piezoelectric transducers or ultrasonic transducers or other aerosol generators.

To obtain the desired particle size of the crystalline steroid solvate the size of the droplets and the amount of steroid in the solvent are varied and controlled. The process is to a certain extent empirical as different systems operating under similar conditions will produce different end particle sizes. However, the droplets should generally be micron sized, say in the range 1-100 microns, preferably 3-30 microns to yield crystals in the size range 0.5-10 microns.

In embodiments of the invention in which droplets are subjected to drying prior to contact with the non-solvent, the dried particles should generally be micron sized, say in the range up to 10 microns, preferably 0.1-10 microns to yield crystals in the size range 0.5-10 microns. Manipulation of the drying conditions and subsequent ultrasound treatment allows crystals to be formed having predetermined characteristics. Such characteristics may include particle morphology, surface free energy, particle size distribution, desired polymorph and, in terms of isolated particles, flowability, reduced electrostatic and cohesive/adhesive properties.

To obtain more generally spherical crystals it is preferred that the droplets of solvent contain a high proportion of steroid. Solvent evaporates from the solvent droplets in the aerosol and this can be controlled and optimized so that the droplets when they are collected in or combined with the beclomethasone non-solvent contain at least 80%, more preferably at least 90%, more preferably at least 95% steroid by weight of droplet.

Hence by variation of a number of parameters, including % product in the droplets and droplet size, the ultimate crystal particle size can be controlled so that particles within the ranges 0.5-10 microns, preferably 0.5-5 microns are obtained. It is further preferred that the solvate particle size distribution is within the range X₁₀=>0.1 microns, X₅₀=<5 microns and X₉₀=<10 microns, more preferably within the range X₁₀=>0.5 microns, X₅₀=<3 microns and X₉₀=<7 microns and in a particular embodiment of the invention the particle size distribution is within the range X₁₀=>0.5 microns, X₅₀=<3 microns and X₉₀=<5 microns.

Suitable solvents for beclomethasone are alcohols and ketones, in particular low molecular weight ketones, alcohols and halogenated alkanes, specific examples being acetone, ethanol, methanol and dichloromethane. In a preferred embodiment of the invention the solvent is or comprises methanol.

The non-solvent should dissolve a very low amount of the steroid, preferably not more than 0.1% w/w; it may be miscible with the solvent and an emulsifier or other agent may be added to aid stability of the droplets suspension. Suitable non-solvents include C₅-C₇ hydrocarbons that can be non-cyclic, straight or branched. A preferred non-solvent is heptane and in a specific embodiment of the invention the heptane is n-heptane. In an alternative embodiment of the invention the non-solvent is water.

Crystallization is effected or initiated by applying ultrasound to the steroid. Crystallization is also effected or initiated by applying ultrasound to the solvate. The ultrasound may be applied continuously or discontinuously such as in a pulsed manner. It may be applied using a variety or devices, such as a probe inserted into the suspension.

Whilst the frequency and amplitude may vary, beclomethasone may be crystallized in the presence of ultrasound having frequency from 20 kHz to 5 MHz. Separately, ultrasound may have an intensity of 0.2 W/cm² or higher, or 0.3 W/cm² or higher.

In embodiments of the invention an ultrasound frequency of 16 kHz to 1 MHz can be used.

The method of the invention typically further comprises drying the solvate particles. Suitable drying methods include spray drying and drying by super-critical CO₂. In a preferred embodiment of the invention the particles are dried by spray drying.

A specific embodiment of the invention provides a method of preparing a dpi formulation of beclomethasone, comprising:
(a) forming a suspension of (i) droplets containing beclomethasone dissolved in a solvent, in (ii) n-heptane,
(b) applying ultrasound to the droplets to form crystallised beclomethasone particles of 0.5-5 microns,
(c) drying the particles by spray drying and
(d) combining the dried particles with a pharmaceutically acceptable excipient.

A further specific embodiment of the invention provides a method of preparing a dpi formulation of beclomethasone, comprising:
(a) forming a solution of beclomethasone in a solvent,
(b) subjecting the solution to a process selected from the group consisting of rapid precipitation, freeze drying, lyophilisation, rapid expansion of supercritical solution, spray drying or mixtures thereof, wherein the beclomethasone is converted into substantially dry solid material,
(c) optionally isolating the beclomethasone from the liquid or gaseous components of the process of step (b),
(d) treating the beclomethasone from step (b) or (c), with n-heptane,
(e) applying ultrasound to the beclomethasone when it is contact with the n-heptane of step (d) to form crystallised beclomethasone particles of 0.5-5 microns,
(f) drying the particles by spray drying, and
(g) combining the dried particles with a pharmaceutically acceptable excipient.

In a further specific embodiment of the method the solvent comprises methanol.

A yet further specific embodiment of the invention provides a method of preparing a dpi formulation of beclomethasone, comprising:
(a) forming a suspension of (i) droplets containing beclomethasone dissolved in methanol, in (ii) n-heptane,
(b) applying ultrasound to the droplets to form crystallised beclomethasone having a particle size distribution within the range X₁₀=>0.5 microns, X₅₀=<3 microns and X₉₀=<5 microns,
c) drying the particles by spray drying and combining the dried particles with a pharmaceutically acceptable excipient.

A still further specific embodiment of the invention provides a method of preparing a dpi formulation of beclomethasone, comprising:
(a) forming a solution of beclomethasone in a solvent,
(b) subjecting the solution to a process selected from the group consisting of rapid precipitation, freeze drying, lyophilisation, rapid expansion of supercritical solution, spray drying or mixtures thereof, wherein the beclomethasone is converted into substantially dry solid material,
(c) optionally isolating the beclomethasone from the liquid or gaseous components of the process of step (b),
(d) treating the beclomethasone from step (b) or (c), with n-heptane,
(e) applying ultrasound to the beclomethasone when it is in contact with the n-heptane of step (d) to form crystallised beclomethasone having a particle size distribution within the range X₁₀=>0.5 microns, X₅₀=<3 microns and X₉₀=<5 microns,
(f) drying the particles by spray drying, and
(g) combining the dried particles with a pharmaceutically acceptable excipient.

An alternative embodiment of the invention provides a method of preparing a dpi formulation of beclomethasone, comprising:
(a) forming a suspension of (i) droplets containing beclomethasone dissolved in a solvent, in (ii) water,
(b) applying ultrasound to the droplets to form crystallised beclomethasone particles of 0.5-5 microns,
(c) drying the particles by spray drying and combining the dried particles with a pharmaceutically acceptable excipient.

A further alternative embodiment of the invention provides a method of preparing a dpi formulation of beclomethasone, comprising:
(a) forming a solution of beclomethasone in a solvent,
(b) subjecting the solution to a process selected from the group consisting of rapid precipitation, freeze drying, lyophilisation, rapid expansion of supercritical solution, spray drying or mixtures thereof, wherein the beclomethasone is converted into substantially dry solid material,
(c) optionally isolating the beclomethasone from the liquid or gaseous components of the process of step (b),
(d) treating the beclomethasone from step (b) or (c), with water,
(e) applying ultrasound to the beclomethasone when it is in contact with the water of step (d) to form crystallised beclomethasone particles of 0.5-5 microns,
(f) drying the particles by spray drying, and
(g) combining the dried particles with a pharmaceutically acceptable excipient.

Reference herein to beclomethasone is reference to the drug substance in any of its suitable and available forms, including salts and other derivatives thereof. Reference to beclomethasone includes but is not limited to beclomethasone dipropionate and beclomethasone valerate, etc.

### EXAMPLES

### Example 1

Beclomethasone was crystallized utilizing ultrasound. Briefly, this method comprised formation of a drug substance solution followed by its atomization, controlled evaporation of the solvent, by spray drying, resulting in substantially amorphous particles, which were then contacted with a non-solvent of beclomethasone and subjected to ultrasound to form a product slurry comprising crystallized beclomethasone particles. The product slurry was then transferred to solid isolation, by spray-drying or supercritical carbon dioxide drying. The method was carried out by Prosonix Ltd of Oxford, UK and further details of this method are as described in WO 2010/007447.

Ultrasound Processing with n-heptane

Protocol:
Input: 6 g of anhydrous beclomethasone diproprionate (BDP)
3% w/v solution of anhydrous BDP in methanol was atomized, spray dried and sonoprocessed in n-heptane
Temperature: 0° C.
Particles were isolated by spray drying

Differential scanning caalorimetry (DSC) and TGA following isolation by spray drying confirmed that the isolated material was an n-heptane solvate and highly crystalline.

SEM showed very homogeneous particles with smooth surfaces and well defined pebble-like morphology. Dry Sympatec PSD analysis confirmed that the particle size distribution was extremely promising and within the inhalation range.

Table 1 shows the results of dry Sympatec PSD analysis:

| Cumulative | |
|---|---|
| distribution | Particle |
| Q3 (%) | Size (μ) |
| X₁₀ | 0.69 |
| X₅₀ | 2.41 |
| X₉₀ | 4.67 |

In order to evaluate the effect of humidity on prolonged storage, processed BDP heptane solvate was subjected to 20% relative humidity (RH) for 48 hours.

DVS mass plot of the processed BDP heptane showed that the material maintained full stability in terms of change of mass.

The sample recovered after storage was analysed by DSC, TGA, PSD and SEM.

The DSC trace of the stored sample indicated no variation in the thermal behaviour of the sample post-humidity treatment.

PSD showed no significant variation of particle size and SEM analysis showed identical morphology to the pre-storage sample.

Table 2 shows the results of dry Sympatec PSD analysis of the post-storage sample:

| Cumulative | |
|---|---|
| distribution | Particle |
| Q3 (%) | Size (μ) |
| X₁₀ | 0.68 |
| X₅₀ | 2.44 |
| X₉₀ | 5.29 |

### Example 2

Beclomethasone is crystallized utilizing ultrasound. Briefly, this method comprises formation of a drug substance solution followed by its atomization, controlled evaporation of the solvent, collection of the pre-concentrated viscous droplets in a vessel containing non-solvent and crystallisation via nucleation with power ultrasound. The product slurry is then transferred to solid isolation, by spray-drying or supercritical carbon dioxide drying. Further details of this method are as described in WO 2004/073827.

Ultrasound Processing with n-heptane

Protocol:
Input: 6 g of anhydrous beclomethasone diproprionate (BDP)
3% w/v solution of anhydrous BDP in methanol is atomized and sonoprocessed in n-heptane
Temperature: 0° C.
Particles are isolated by spray drying

### Example 3

Ultrasound processing was carried out as described in Example 1 above.

Beclomethasone hydrate Obtained by Crystallization in the Presence of Ultrasound

Protocol:
Input: 6 g of anhydrous beclomethasone diproprionate (BDP)
3% w/v solution of anhydrous BDP in methanol was atomized, spray dried and sonoprocessed in water
Temperature: 0° C.
Particles were isolated by spray drying

Differential scanning caalorimetry (DSC) and TGA following isolation by spray drying showed highly crystalline BDP hydrate.

SEM showed particles with smooth surfaces and homogeneous morphology. Dry Sympatec PSD analysis confirmed that the particle size distribution was well within the inhalation range.

Table 3 shows the results of dry Sympatec PSD analysis:

| Cumulative | |
|---|---|
| distribution | Particle |
| Q3 (%) | Size (μ) |
| X₁₀ | 0.51 |
| X₅₀ | 1.35 |
| X₉₀ | 3.17 |

In order to evaluate the effect of humidity on prolonged storage processed BDP hydrate was subjected to 20% relative humidity (RH) for 48 hours.

DVS mass plot of the processed BDP hydrate showed that during storage the sample initially underwent considerable weight loss due to partial dehydration.

The sample achieved a steady state after about 1500 minutes. The loss of water from the sample is likely to reflect the loss of free water remaining in the sample after spray drying, as this drying technique is usually not 100% efficient.

These results indicate that BDP formed a hydrate at a very low moisture content, and is anticipated to retain stability on prolonged storage.

The sample recovered after storage was analysed by DSC, TGA, PSD and SEM.

The DSC trace of the stored sample indicated no variation in the thermal behavior of the sample post-humidity treatment.

PSD showed no significant variation of particle size and SEM analysis showed identical morphology to the pre-storage sample.

Table 4 shows the results of dry Sympatec PSD analysis of the post-storage sample:

| Cumulative | |
|---|---|
| distribution | Particle |
| Q3 (%) | Size (μ) |
| X₁₀ | 0.51 |
| X₅₀ | 1.37 |
| X₉₀ | 2.95 |

### Example 4

Ultrasound processing is carried out as described in Example 2 above.

Beclomethasone hydrate Obtained by Crystallization in the Presence of Ultrasound

Protocol:
Input: 6 g of anhydrous beclomethasone diproprionate (BDP)
3% w/v solution of anhydrous BDP in methanol is atomized and sonoprocessed in water
Temperature: 0° C.
Particles are isolated by spray drying

### Example 5

### Beclomethasone Formulation

A beclomethasone dpi formulation is prepared, by dissolving beclomethasone in a solvent and then forrning a suspension of the beclomethasone solution in a non-solvent, and crystallizing the beclomethasone by application of ultrasound, as described in WO 2004/073827.

The operating parameters including flow rate and ultrasound power are varied so as to obtain a particle size for crystallized beclomethasone substantially within the size range 2-3 microns.

The beclomethasone solvate obtained is formulated with carriers then subjected to end-sterilization by irradiation to yield end formulations to be dispensed in the following individual doses:

| | | | |
|---|---|---|---|
| 1 | Beclomethasone | 25 | mcg |
| | Lactose | 1.25 | mg |
| 2 | Beclomethasone | 50 | mcg |
| | Lactose | 2.5 | mg |
| 3 | Beclomethasone | 125 | mcg |
| | Lactose | 6.35 | mg |
| 4 | Beclomethasone | 37.5 | mcg |
| | Lactose | 1.25 | mg |
| 5 | Beclomethasone | 75 | mcg |
| | Lactose | 2.5 | mg |
| 6 | Beclomethasone | 187.5 | mcg |
| | Lactose | 6.35 | mg |
| 7 | Beclomethasone | 25 | mcg |
| | Mannitol | 1.25 | mg |
| 8 | Beclomethasone | 0.5 | mcg |
| | Mannitol | 2.5 | mg |
| 9 | Beclomethasone | 125 | mcg |
| | Mannitol | 6.35 | mg |
| 10 | Beclomethasone | 37.5 | mcg |
| | Mannitol | 1.25 | mg |
| 11 | Beclomethasone | 75 | mcg |
| | Mannitol | 2.5 | mg |
| 12 | Beclomethasone | 187.5 | mcg |
| | Mannitol | 6.35 | mg |

### Example 6

### Beclomethasone Formulation

A beclomethasone dpi formulation is prepared, by dissolving beclomethasone in a solvent and evaporating the solvent by spray drying under controlled conditions, resulting in substantially amorphous particles, which are then contacted with a non-solvent of beclomethasone, and crystallizing the beclomethasone by application of ultrasound, as described in WO 2010/007447.

The operating parameters including flow rate and ultrasound power are varied so as to obtain a particle size for crystallized beclomethasone substantially within the size range 2-3 microns.

The beclomethasone solvate obtained is formulated with carriers then subjected to end-sterilization by irradiation to yield end formulations to be dispensed in the following individual doses:

| | | | |
|---|---|---|---|
| 1 | Beclomethasone | 25 | mcg |
| | Lactose | 1.25 | mg |
| 2 | Beclomethasone | 50 | mcg |
| | Lactose | 2.5 | mg |
| 3 | Beclomethasone | 125 | mcg |
| | Lactose | 6.35 | mg |
| 4 | Beclomethasone | 37.5 | mcg |
| | Lactose | 1.25 | mg |
| 5 | Beclomethasone | 75 | mcg |
| | Lactose | 2.5 | mg |
| 6 | Beclomethasone | 187.5 | mcg |
| | Lactose | 6.35 | mg |
| 7 | Beclomethasone | 25 | mcg |
| | Mannitol | 1.25 | mg |
| 8 | Beclomethasone | 0.5 | mcg |
| | Mannitol | 2.5 | mg |
| 9 | Beclomethasone | 125 | mcg |
| | Mannitol | 6.35 | mg |
| 10 | Beclomethasone | 37.5 | mcg |
| | Mannitol | 1.25 | mg |
| 11 | Beclomethasone | 75 | mcg |
| | Mannitol | 2.5 | mg |
| 12 | Beclomethasone | 187.5 | mcg |
| | Mannitol | 6.35 | mg |

### Comparative Examples

Ultrasound processing was carried out as described in Example 1 above.

### Comparative Example 1

Ultrasound Processing with Cyclopentane

Protocol:
Input: 2 g of anhydrous beclomethasone diproprionate (BDP)
3% w/v solution of anhydrous BDP in methanol in methanol was atomized, spray dried and sonoprocessed in cyclopentane
Temperature: 0° C.
Particles were isolated by supercritical CO₂

Microscope imaging of the suspension prior to isolation showed partially agglomerated particles up to 5 μm.

Differential scanning caalorimetry (DSC) following isolation by supercritical CO₂ showed evidence for amorphous BDP but no evidence for a BDP solvate.

SEM showed particles with smooth surfaces and homogeneous morphology but also large clusters up to 10 μm in size. Dry Sympatec PSD analysis confirmed the presence of mostly ˜4 μm particles alongside larger clusters or agglomerates larger than 20 μm.

Table 5 shows the results of dry Syrnpatec PSD analysis:

| Cumulative | |
|---|---|
| distribution | Particle |
| Q3 (%) | Size (μ) |
| X₁₀ | 0.84 |
| X₅₀ | 3.58 |
| X₉₀ | 8.76 |

### Comparative Example 2

Ultrasound Processing with Cyclohexane

Protocol:
Input: 2 g of anhydrous beclomethasone diproprionate (BDP)
3% w/v solution of anhydrous BDP in methanol was atomized, spray dried and sonoprocessed in cyclohexane
Temperature: 0° C.
Particles were isolated by supercritical CO₂

Microscope imaging of the suspension prior to isolation showed partially agglomerated particles up to 10 μm.

Differential scanning calorimetry (DSC) and TGA following isolation by supercritical CO₂ showed evidence for highly crystalline anhydrous BDP and no evidence for a solvate.

SEM images showed particles with a smooth surface and very homogenous specific diamond-shaped morphology that were up to 10 μm in size. No significant increases in particle size were observed during supercritical CO₂ isolation.

Table 6 shows the results of dry Sympatec PSD analysis:

| Cumulative | |
|---|---|
| distribution | Particle |
| Q3 (%) | Size (μ) |
| X₁₀ | 1.78 |
| X₅₀ | 8.10 |
| X₉₀ | 15.10 |

The invention thus provides beclomethasone-containing dpi formulations and methods for the manufacture thereof.

1-26. (canceled)

27. A Dry Powder Inhaler (DPI) formulation comprising a solvate of (i) beclomethasone and (ii) heptane.

28. The DPI formulation of claim 27, wherein the heptane is n-heptane.

29. The DPI formulation of claim 27, wherein the solvate is in crystalline form.

30. The DPI formulation of claim 27, wherein the solvate is obtained by crystallization in the presence of ultrasound.

31. The DPI formulation of claim 27, comprising particles of diameter 0.5-10 microns.

32. The DPI formulation of claim 31, wherein the particle size distribution is within the range X₁₀≧0.5 microns, X₅₀≦3 microns and X₉₀≦5 microns.

33. The DPI formulation of claim 27, further comprising a beta-agonist.

34. The DPI formulation of claim 33, wherein the beta-agonist is salbutamol, levalbuterol or formoterol.

35. The DPI formulation of claim 27, further comprising a carrier.

36. The DPI formulation of claim 35, wherein the carrier is lactose, mannitol, glucose or polyethylene glycol.

37. A method of preparing a solvate of beclomethasone, comprising forming a suspension of (i) droplets containing beclomethasone dissolved in a solvent, in (ii) heptane, and applying ultrasound to the droplets to form the solvate.

38. The method according to claim 37, wherein the heptane is n-heptane.

39. The method according to claim 37, further comprising drying the solvate particles by spray drying.

40. The method according to claim 37, wherein the solvate is in crystalline form.

41. A method of preparing a DPI formulation of beclomethasone comprising:
(a) forming a suspension of (i) droplets containing beclomethasone dissolved in a solvent, in (ii) n-heptane, (b) applying ultrasound to the droplets to form crystallized beclomethasone particles of 0.5-5 microns,
(c) drying the particles by spray drying and
(d) combining the dried particles with a pharmaceutically acceptable excipient.

42. The method according to claim 41, wherein the solvent comprises methanol.

43. A method of preparing a DPI formulation of beclomethasone comprising:
(a) forming a suspension of (i) droplets containing beclomethasone dissolved in methanol, in (ii) n-heptane,
(b) applying ultrasound to the droplets to form crystallized beclomethasone having a particle size distribution within the range X₁₀≧0.5 microns, X₅₀≦3 microns and X₉₀≦5 microns,
(c) drying the particles by spray drying and
(d) combining the dried particles with a pharmaceutically acceptable excipient.
